(19) United States
(12) Patent Application Publication
(10) Pub. No.: US 2026/0182979 A1
(43) Pub. Date: Jul. 2, 2026

(54) ENDOSCOPIC TREATMENT METHOD

(71) Applicant: OLYMPUS CORPORATION, Tokyo (JP)

(72) Inventors: Kosuke IKEDA, Fussa-shi (JP); Kunihide Kaji, Hachioji-shi (JP)

(73) Assignee: OLYMPUS CORPORATION, Tokyo (JP)

(21) Appl. No.: 19/002,042

(22) Filed: Dec. 26, 2024

(51) Int. Cl. A61B 17/02; A61B 17/00; A61B 17/04; A61B 18/00; A61B 18/14

(52) U.S. Cl. CPC A61B 17/0281; A61B 17/0469; A61B 18/1482; A61B 2017/00269; A61B 2018/00494; A61B 2018/00595; A61B 2018/00601; A61B 2018/00982

(57) ABSTRACT

Provided is an endoscopic treatment method for resecting a full thickness of a gastrointestinal wall under an endoscope, the method including: making a full circumferential incision in tissue around a target tissue up to a submucosal layer; fixing a traction device to a first location positioned further outside the target tissue; pulling the first location using the traction device so that a first region and a second region are folded and approach each other along a fold line that passes through the first location and divides the target tissue into the first region and the second region, to lift the full thickness of the gastrointestinal wall including the target tissue toward a gastrointestinal lumen side; suturing the submucosal layer exposed by full circumferential incision; and resecting the target tissue placed in the lumen side of the sutured area.

### TECHNICAL FILED

The present disclosure relates to an endoscopic treatment method.

### BACKGROUND

In endoscopic full-thickness resection (EFTR), there is a need to reliably resect and suture the full thickness of a tubular wall from a lesion portion to the serosa. When the full thickness of a tubular wall is resected by an endoscope having a cap-type device attached to its distal end as described in Patent Document 1, it is difficult to visually recognize and check an area to be resected under the endoscope.

### CITATION LIST

### Patent Document

[Patent Document 1] U.S. Pat. No. 11,540,697

### SUMMARY

An endoscopic treatment method according to a first aspect of the present disclosure is a method for resecting a full thickness of a gastrointestinal wall under an endoscope, the method including: making a full circumferential incision in tissue around a target tissue up to a submucosal layer; fixing a traction device to a first location positioned further outside the target tissue; pulling the first location using the traction device so that a first region and a second region are folded and approach each other along a fold line that passes through the first location and divides the target tissue into the first region and the second region, to lift the full thickness of the gastrointestinal wall including the target tissue toward a gastrointestinal lumen side; suturing the submucosal layer exposed by full circumferential incision; and resecting the target tissue placed in the lumen side of the sutured area.

### BRIEF DESCRIPTION OF THE DRAWINGS

FIG. 1 An overall view showing an endoscope.

FIG. 2 An overall view showing a needle holder.

FIG. 3 A perspective view showing a gripping portion of the same needle holder.

FIG. 4 An overall view showing a high frequency knife.

FIG. 5 A perspective view of a distal portion of the same high frequency knife.

FIG. 6 A view showing a marking step in an endoscopic treatment method according to a first embodiment.

FIG. 7 A view showing the same marking step.

FIG. 8 A view showing a full circumferential incision step.

FIG. 9 A view showing the same full circumferential incision step.

FIG. 10 A view showing the same full circumferential incision step.

FIG. 11 A view showing a fixing step.

FIG. 12 A view showing the same fixing step.

FIG. 13 A view showing the same fixing step.

FIG. 14 A view showing the same fixing step.

FIG. 15 A view showing a lifting step.

FIG. 16 A view showing the same lifting step.

FIG. 17 A cross-sectional view showing a lifted target tissue.

FIG. 18 A view showing a suture step.

FIG. 19 A view showing a planned suture area being pressed during a suture step.

FIG. 20 A view showing the same planned suture area that has been sutured.

FIG. 21 A view showing a fixing step and a lifting step in an endoscopic treatment method according to a second embodiment.

FIG. 22 A flowchart of an endoscopic treatment method according to a third embodiment.

### DETAILED DESCRIPTION

### First Embodiment

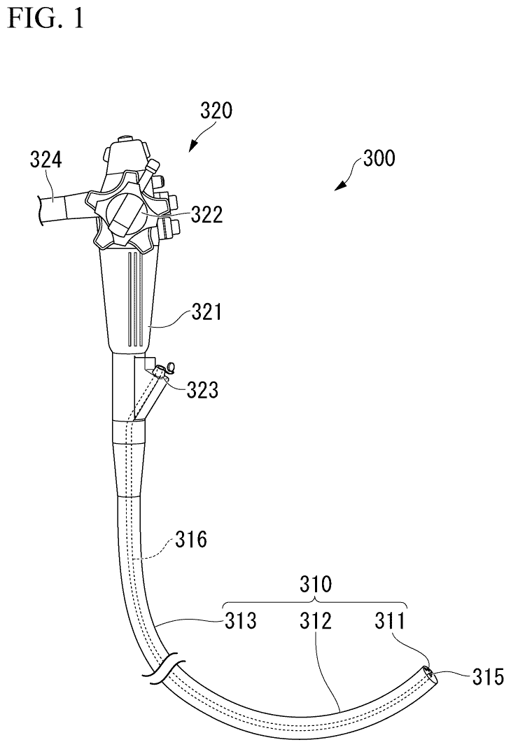

Endoscopic treatment according to a first embodiment of the present disclosure will be described with reference to FIGS. 1 to 20. An endoscope 300, a needle holder 200, a high frequency knife 100, a needle-attached suture 400, and a linear stapler 500 are used for the endoscopic treatment according to the present embodiment.

FIG. 1 is an overall view showing the endoscope 300.

The endoscope 300 is a well-known flexible endoscope and includes an insertion unit 310 inserted into the body from a distal end and an operation unit 320 attached to a proximal end of the insertion unit 310.

The insertion unit 310 has an imaging unit 311, a curved portion 312, and a flexible unit 313. A channel 316 is provided inside the insertion unit 310 for inserting a treatment tool such as the needle holder 200 or the high frequency knife 100. A distal opening 315 of the channel 316 is provided at the distal end of the insertion unit 310.

The imaging unit 311 includes, for example, an imaging element such as a CCD or a CMOS and can image a site to be treated (hereinafter also referred to as a “target site”). The curved portion 312 is curved according to an operation of the operation unit 320 by an operator. The flexible unit 313 is a tubular part having flexibility.

The operation unit 320 is connected to the flexible unit 313. The operation unit 320 has a grip 321, an input unit 322, a proximal opening (forceps port) 323 of the channel 316, and a universal cord 324.

The grip 321 is a part gripped by an operator. The input unit 322 accepts an operation input for curving the curved portion 312. The universal cord 324 outputs an image imaged by the imaging unit 311 to an image processing device.

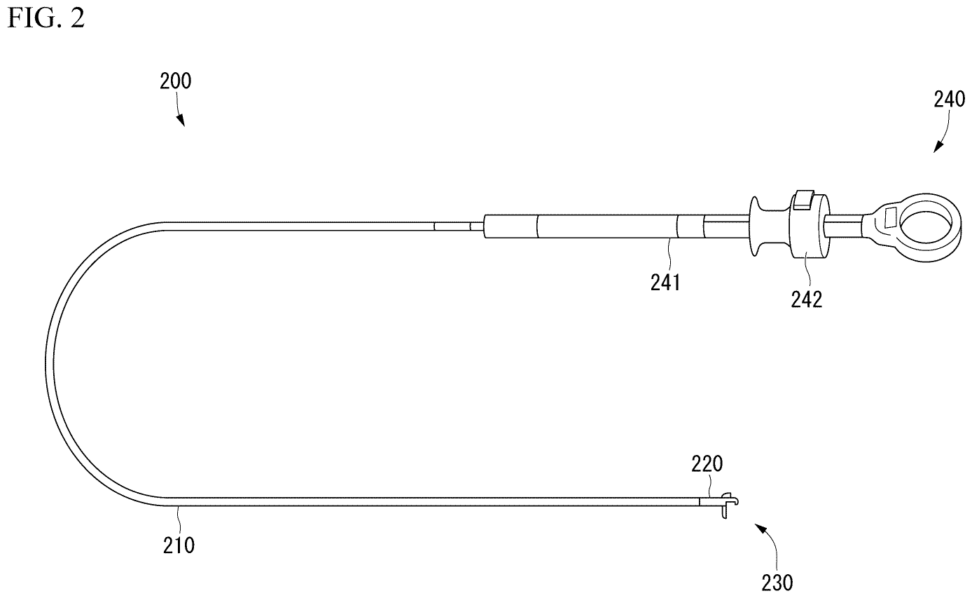

FIG. 2 is an overall view showing the needle holder 200.

The needle holder 200 includes a sheath 210, a hard portion 220, a gripping portion 230, an operation unit 240, and an operation wire 250 inserted through the sheath 210. The needle holder 200 is inserted into the channel 316 of the endoscope 300 for use.

The sheath 210 is a flexible, long, cylindrical member. The hard portion 220 is provided at a distal end of the sheath 210. The operation unit 240 is provided at a proximal end of the sheath 210.

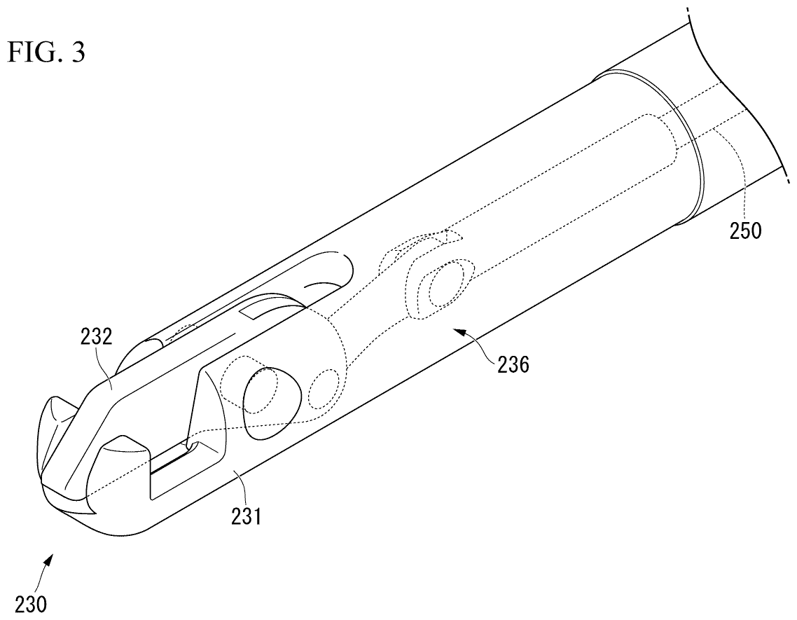

FIG. 3 is a perspective view showing the gripping portion 230 of the needle holder 200.

The gripping portion 230 has a first gripping member 231, a second gripping member 232, and a ring mechanism 236. The first gripping member 231 and the second gripping member 232 are configured to allow opening and closing actions.

The operation unit 240 has a main body 241 and a slider 242. The slider 242 is linked to the main body 241 so as to be advanceable and retractable. The operation wire 250 extending from the sheath 210 is connected to the slider 242 through the interior of the main body 241.

The operation wire 250 is disposed inside the sheath 210 along a longitudinal axis of the sheath 210. A practitioner advances and retracts the slider 242 along the main body 241, causing the operation wire 250 to advance and retract. The gripping portion 230 opens and closes as the operation wire 250 advances and retracts.

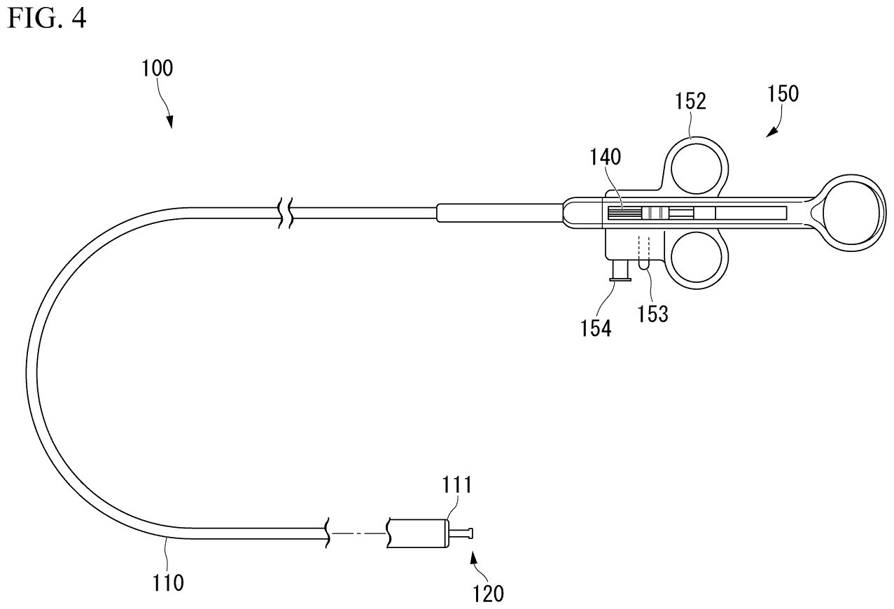

FIG. 4 is an overall view showing the high frequency knife 100.

The high frequency knife 100 is a high frequency dual knife capable of, for example, water supply, incision, cauterization, and detachment. The high frequency knife 100 includes a sheath 110, a knife 120, an operation wire 140, and an operation unit 150. The high frequency knife 100 is inserted into the channel 316 of the endoscope 300 for use.

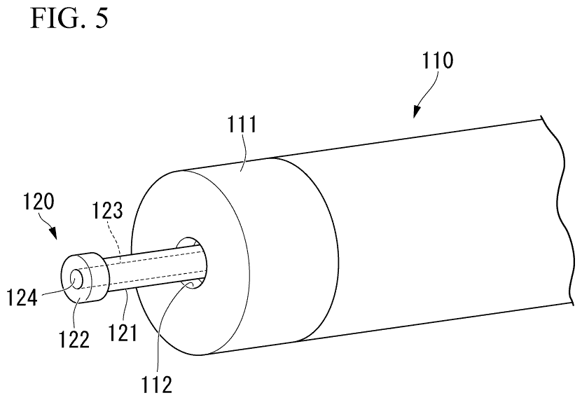

FIG. 5 is a perspective view of a distal portion of the high frequency knife 100.

The sheath 110 is a flexible, insulating, long, cylindrical member. An insulating distal tip 111 having a through-hole 112 penetrating in the longitudinal direction is attached to a distal end of the sheath 110.

The knife (electrode, rod) 120 is a metallic elongated member. The knife 120 is formed of a material such as stainless steel, for example. The knife 120 has a knife main body 121 and a flange 122.

The knife main body 121 is a metallic round rod-shaped member. The operation wire 140 is attached to a proximal end of the knife main body 121. When a high frequency current is supplied from the operation wire 250 to the knife 120, the knife main body 121 and the flange 122 function as monopolar electrodes that output the high frequency current to biological tissues.

The flange (distal portion) 122 is an annular plate-shaped conductive member provided at a distal end of the knife main body 121. The width of the flange 122 in the radial direction perpendicular to the longitudinal direction is longer than the width of the knife main body 121 in the radial direction.

The knife main body 121 and the flange 122 have a water supply conduit 123. The water supply conduit 123 communicates with a distal opening 124 formed in the flange 122.

The operation wire 140 is a hollow coil wire through which the sheath 110 is inserted. A distal end of the operation wire 140 is connected to the knife 120, and a proximal end of the operation wire 140 is connected to a slider 152 of the operation unit 150.

The operation unit 150 has an operation unit main body 151, the slider 152, a power supply connector 153, and a liquid supply port 154.

The slider 152 is movably mounted along the operation unit main body 151. The proximal end of the operation wire 140 is attached to the slider 152. As a practitioner advances and retracts the slider 152 with respect to the operation unit main body 151, the operation wire 140 and the knife 120 advance and retract.

The power supply connector 153 is connectable to a high frequency power supply device and is connected to the proximal portion of the operation wire 140 via a conductive wire. The power supply connector 153 can supply a high frequency current supplied from the high frequency power supply device to the knife 120 via the operation wire 140.

A fluid (such as a liquid chemical) supplied from the liquid supply port 154 passes through a hollow portion of the operation wire 140 and the water supply conduit 123 of the knife 120 and is discharged from the distal opening 124.

A high frequency knife having no water supply function may be used instead of the high frequency knife 100. In that case, a treatment tool having a water supply function such as a local injection needle having a water supply function is used together.

Next, the endoscopic treatment according to the present embodiment will be described. Specifically, a full-thickness resection procedure of a lesion portion in endoscopic full-thickness resection (EFTR) will be described.

As a preparatory operation, a practitioner identifies a lesion portion by a well-known method. Specifically, the practitioner inserts the insertion unit 310 of the endoscope 300 into the gastrointestinal tract (for example, the esophagus, the stomach, the duodenum, and the colon) and identifies a lesion portion TU while observing an image obtained by the imaging unit 311 of the endoscope 300.

### marking Step

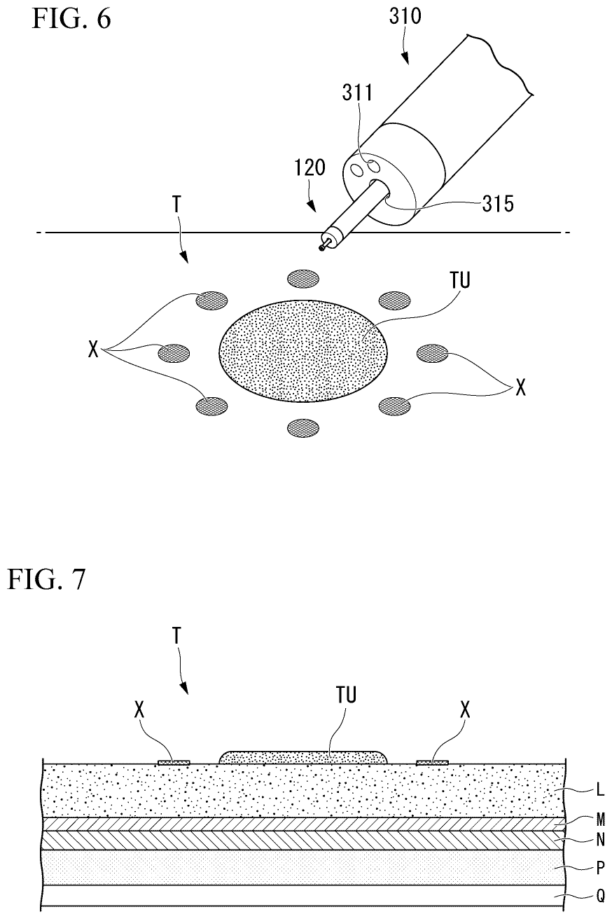

FIGS. 6 and 7 are views showing a marking step.

A practitioner inserts the high frequency knife 100 into the channel 316 to cause the knife 120 provided at the distal end of the high frequency knife 100 to protrude from the distal opening 315 of the endoscope 300. The practitioner presses the knife 120 of the high frequency knife 100 against periphery tissue surrounding the periphery of the lesion portion TU which is a target tissue T for cauterization, thereby attaching a marking X. The marking step is not essential, and the practitioner may omit the marking step.

### Full Circumferential Incision Step

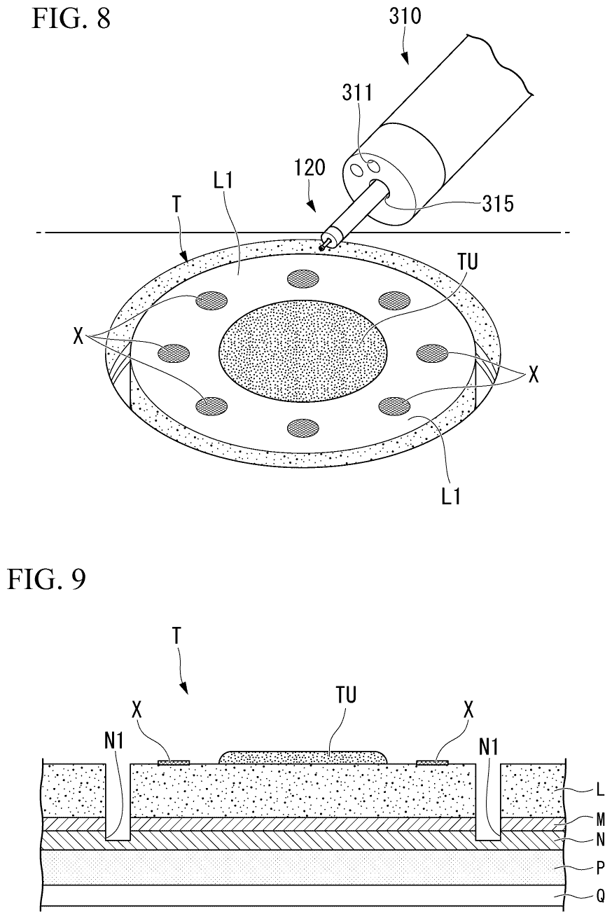

FIGS. 8 to 10 are views showing a full circumferential incision step.

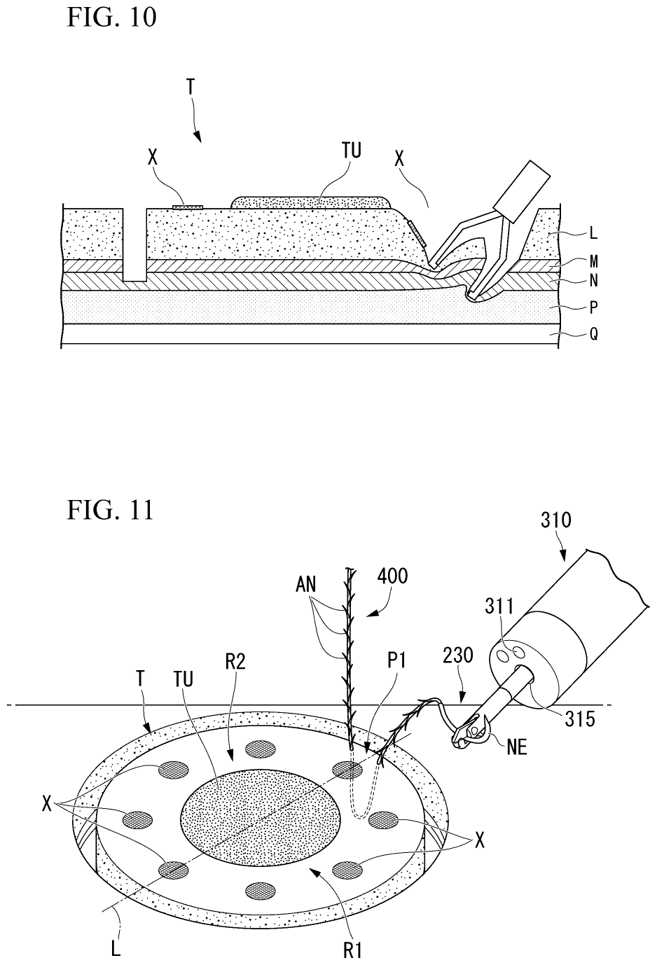

A practitioner moves the knife 120 while energizing the knife 120 with a high frequency current and makes an incision around the lesion portion TU over the entire circumference. The practitioner makes a full circumferential incision in the tissue around the lesion portion TU and outside the marked areas up to a submucosal layer N with the knife 120 provided at the distal end of the high frequency knife 100. The practitioner may perform a local injection on around the lesion portion TU as necessary. At least a part of the full circumferential incision site becomes a planned suture area (planned suture line) R3. As shown in FIG. 10, the practitioner may prevent deviation of a mucosal layer L (for example, L1 shown in FIG. 10) on the resection side by gripping the mucosal layer L and at least one or more of layers below the mucosal layer L with a clip CL or suturing it with a needle thread, for example. For example, the follower fixes a muscle layer P and the peripheral mucosa of the marked areas with the clip CL or with a suture. The practitioner may trim the submucosal layer N (for example, N1 shown in FIG. 9) on the side remaining in the body to secure a space for inputting a suture device. The practitioner may omit the full circumferential incision step or may make an incision only in a portion of the submucosal layer N that needs to be exposed, rather than the entire circumference.

### Fixing Step

FIGS. 11 to 14 are views showing a fixing step.

A practitioner fixes a traction device to a first location P1. In the present embodiment, the traction device is the needle-attached suture 400. The needle-attached suture 400 has a suture (connection member) S, a suture needle (penetration member) NE provided at a distal end of the suture S, and a fixation tag TG provided at a proximal end of the suture S. The suture needle NE used in the present embodiment is a curved needle. The suture S is provided with a barb (anchor) AN extending toward the proximal side. The traction device is not limited to the needle-attached suture 400, and may be other treatment tools that combines, for example, a clip (gripping member) with a suture or an elastic body such as rubber.

The practitioner removes the high frequency knife 100 from the channel 316 of the endoscope 300 and inserts the needle holder 200 into the channel 316. The practitioner causes the gripping portion 230 of the needle holder 200 to protrude from the distal opening 315 of the endoscope 300. The practitioner grips the suture needle NE of the needle-attached suture 400 by the gripping portion 230.

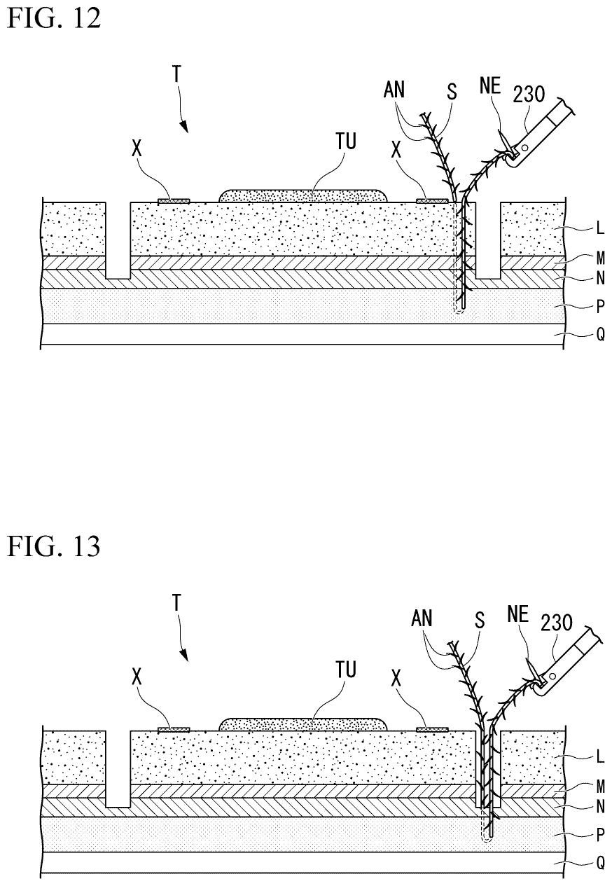

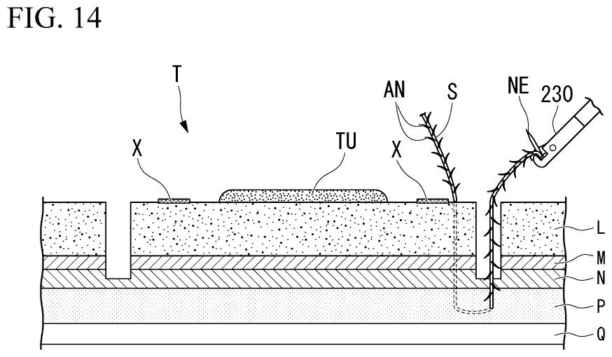

The practitioner sews and fixes the needle-attached suture 400 to the first location P1 which is positioned further inside the part of the full circumferential incision site and further outside the lesion portion TU. The practitioner pierces the needle-attached suture 400 at least to the depth of the muscle layer P to penetrate the muscle layer P. The first location P1 is positioned proximal to the lesion portion TU when viewed from the endoscope 300. As shown in FIG. 13, the first location P1 may be the part of the full circumferential incision site where the submucosal layer N is exposed. The practitioner cause the needle-attached suture 400 to penetrate the muscle layer P. At this time, as shown in FIG. 14, the practitioner may prevent the mucosal layer L from deviation by passing the needle-attached suture 400 through the mucosal layer L inside the full circumferential incision in addition to the muscle layer P.

### Lifting Step

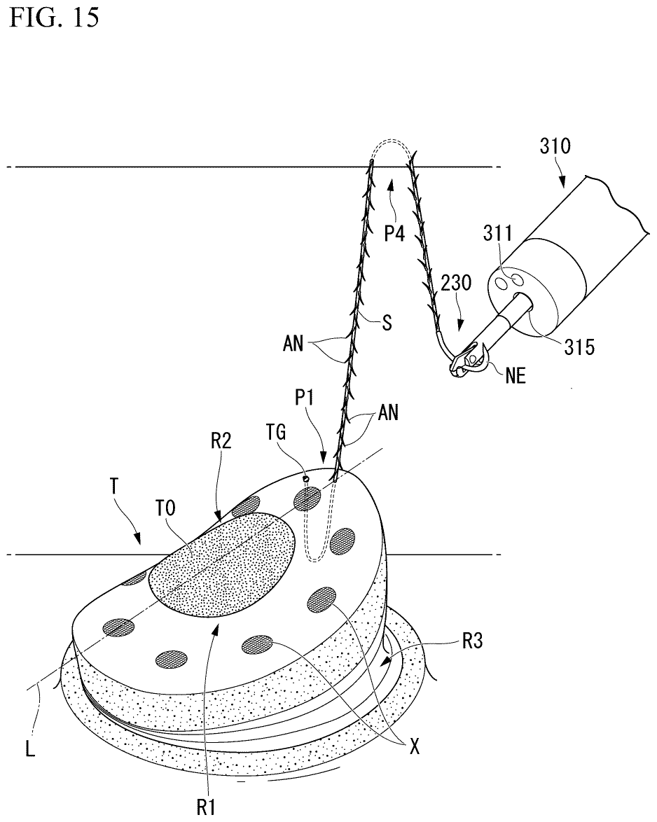

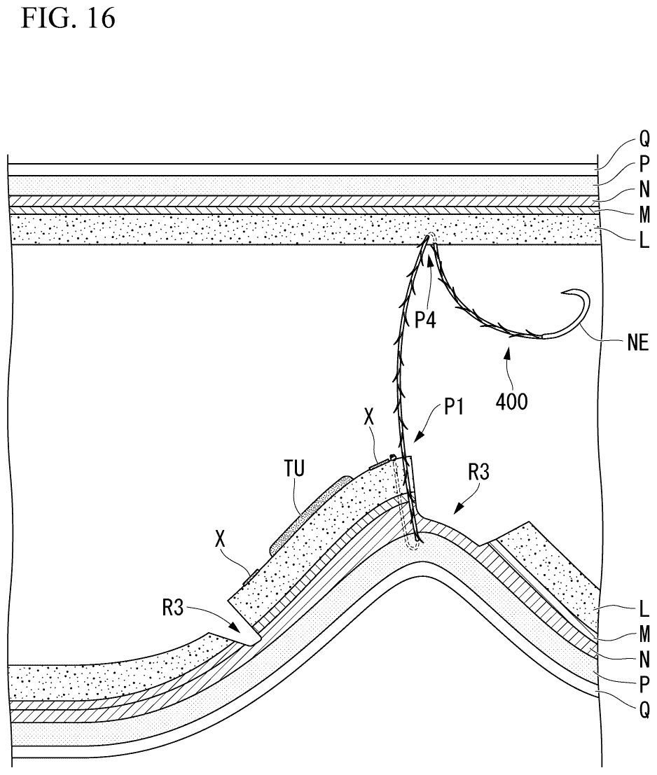

FIGS. 15 and 16 are views showing a lifting step.

A line that passes through the first location P1 and divides the lesion portion TU into a first region R1 and a second region R2 is called a “fold line (reference line) L.” The fold line L is desirably set so that the first region R1 and the second region R2 have approximately the same area.

A practitioner pulls the first location P1 with the needle-attached suture 400 and lifts the full thickness of the gastrointestinal wall including the lesion portion TU toward a gastrointestinal lumen side. The first region R1 and the second region R2 are folded and approach each other. Since the needle-attached suture 400 penetrates the muscle layer P, the mucosal layer L is less likely to be deviated with respect to the muscle layer P.

The practitioner sews and fixes the needle-attached suture 400 to a fourth location P4 in the tissue of the gastrointestinal wall facing the target tissue T. Since the suture S is provided with the barb (anchor) AN, it does not escape from the fourth location P4. Since the fixation tag (stopper) TG is provided at the proximal end of the suture S, the suture S does not escape from the first location P1 and is fixed to the first location P1. As a result, the first location P1 is connected to the fourth location P4 through the suture (connection member) S of the needle-attached suture 400. The fixation tag TG is desirably formed in a loop shape, and the suture S is more reliably fixed to the first location P1 by passing the suture S sewn at the first location P1 through the loop-shaped fixation tag TG. The fourth location P4 is desirably placed at a position facing the first location P1.

The practitioner pulls the suture (connection member) S to apply tension to the suture S connecting the first location P1 to the fourth location P4. For example, the practitioner pulls the suture needle NE using the needle holder 200 to apply tension to the suture S. For example, the practitioner applies tension to the suture S by delivering air endoscopically and dilating the lumen.

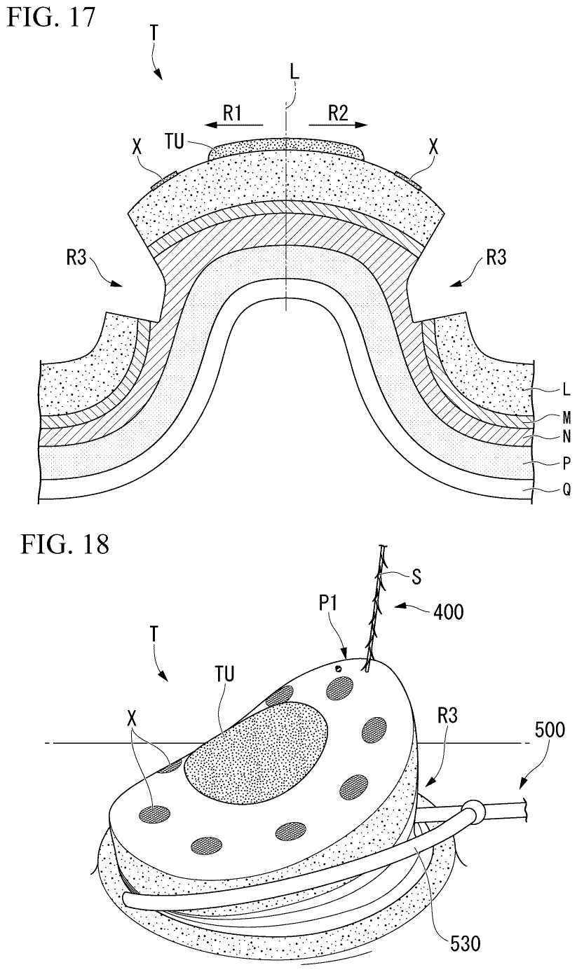

FIG. 17 is a cross-sectional view showing a lifted target tissue T.

The practitioner applies tension to the suture S to lift the full thickness of the gastrointestinal wall including the target tissue T toward the gastrointestinal lumen side. The practitioner lifts the full thickness of the gastrointestinal wall including a serous membrane Q to bring the serous membrane Q of the first region R1 and the serous membrane Q of the second region R2 closer together. When the practitioner lifts the target tissue T, it is desirable to lift it to a position where the muscle layer P of the target tissue T is sufficiently retracted to the gastrointestinal lumen side. By lifting the target tissue T, the planned suture area R3 can be placed in the gastrointestinal lumen, and the practitioner is likely to visually recognize the planned suture area R3 with the endoscope 300. The planned suture area R3 is a gastrointestinal wall positioned further outside the target tissue T, and is desirably the submucosal layer N exposed by incision.

### Endoscope Movement Step

A practitioner moves the insertion unit 310 of the endoscope 300 so that an endoscope optical axis is substantially parallel to the fold line L. Compared to a case where the insertion unit 310 of the endoscope 300 is placed so that the endoscope optical axis intersects with the fold line L, the range in which the planned suture area R3 can be visually recognized is widened for the reasons described below. In the case where the insertion unit 310 of the endoscope 300 is placed so that the endoscope optical axis intersects with the fold line L, both the planned suture area R3 on the first region R1 and the planned suture area R3 on the second region R2 cannot be visually recognized at the same time. On the other hand, in the case where the insertion unit 310 of the endoscope 300 is placed so that the endoscope optical axis is substantially parallel to the fold line L, both the planned suture area R3 on the first region R1 and the planned suture area R3 on the second region R2 can be visually recognized at the same time.

Conventionally, it was difficult to visually recognize and check an area to be resected with an endoscope with a cap-type device attached to its distal end as described in Patent Document 1. According to an endoscopic treatment method according to the present embodiment, the planned suture area R3 can be visually recognized by the endoscope 300, and whether or not the planned suture area R3 is placed at a position at which the full thickness of the tubular wall can be resected and sutured can be accurately confirmed. If the planned suture area R3 is not placed at a position at which the full thickness of the tubular wall can be resected and sutured, the practitioner applies tension to the suture S to further lift the full thickness of the gastrointestinal wall including the target tissue T.

### Suture Step

FIG. 18 is a view showing a suture step.

A practitioner removes the needle holder 200 from the channel 316 of the endoscope 300 and inserts a gripping device into the channel 316. The gripping device may be inserted into the gastrointestinal tract independently of the endoscope 300 rather than through the channel 316 of the endoscope 300. In the present embodiment, the gripping device is a linear stapler 500. The linear stapler 500 has a gripping portion 530. The gripping portion 530 sutures a pinched biological tissue with a staple ST and cuts the pinched biological tissue. The gripping device is not limited to the linear stapler 500, and may be, for example, other treatment tools such as a clip.

In the endoscopic treatment in the present embodiment, the needle-attached suture (traction device) 400 and the linear stapler (gripping device) 500 are used. For this reason, the practitioner can perform treatment by moving the linear stapler (gripping device) 500 regardless of the position of the needle-attached suture (traction device) 400 while maintaining the target tissue T lifted by the needle-attached suture (traction device) 400.

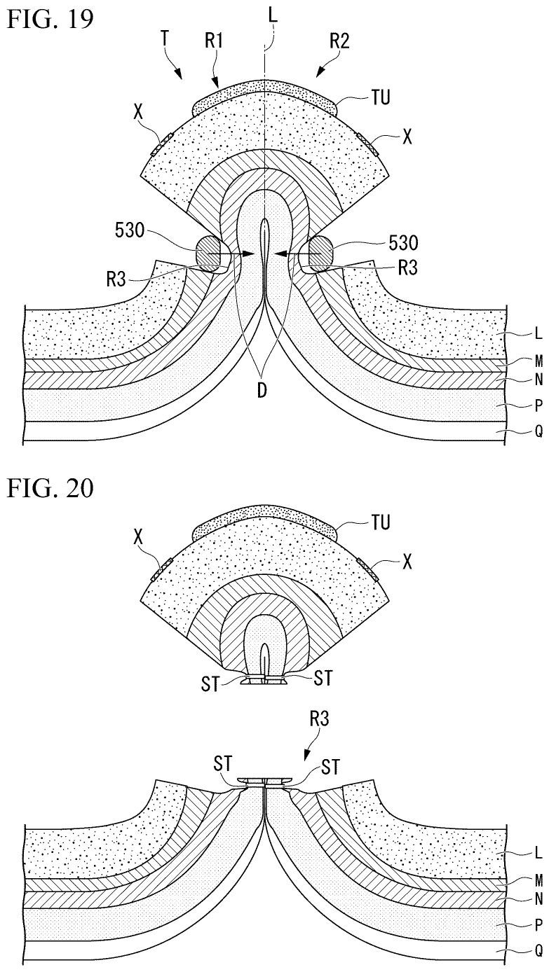

FIG. 19 is a view showing a planned suture area R3 being pressed during a suture step.

The practitioner presses the planned suture area R3 using the gripping portion 530 of the linear stapler 500 after lifting the target tissue T using the needle-attached suture 400 and before suturing the planned suture area R3. Specifically, the practitioner presses the planned suture area R3 so that the first region R1 and the second region R2 are in close contact with each other from a direction D or the like substantially perpendicular to the lifting direction and to the fold line. Accordingly, the serous membrane Q of the first region R1 and the serous membrane Q of the second region R2 are in close proximity. It is desirable for the practitioner to press the planned suture area R3 until the serous membrane Q of the first region R1 and the serous membrane Q of the second region R2 are in close contact with each other. In addition, it is desirable for the practitioner to press the planned suture area R3 sequentially from one side (proximal or distal) toward the other to prevent entanglement of other organs on the serous membrane Q side. It is desirable for the practitioner to increase the pressure in the abdominal cavity by percutaneously delivering air to the abdominal cavity side before pressing to reduce the risk of entanglement of other organs on the serous membrane Q side. The position to be pressed in the planned suture area R3 is desirably the position of the muscle layer P that has been sufficiently retracted to the gastrointestinal lumen side. The practitioner may expel other organs on the serous membrane Q side to other locations by postural change.

FIG. 20 is a view showing the planned suture area R3 that has been sutured.

The practitioner uses the gripping portion 530 of the linear stapler 500 to suture the pinched biological tissue with the staple ST and resect the pinched biological tissue. The full thickness of the gastrointestinal wall including the lesion portion TU and the serous membrane Q to suture the resected portion. If the serous membrane Q of the first region R1 and the serous membrane Q of the second region R2 are kept in close contact with each other, the practitioner can suitably carry out resection and suture of the full thickness of the gastrointestinal wall. If the biological tissue to be resected is large enough not to be sutured and resected at one time, the practitioner repeats the suture operation (including adjustment of lifting volume, pressing, stapling, and the like) multiple times. If the biological tissue to be resected is large enough to be sutured and resected at one time, the practitioner may omit suture of the resected biological tissue using the staple ST. The practitioner may cover a mucosal defect with the surrounding mucosal layer L and close the covered mucosal layer L. It is desirable for the practitioner to close the mucosal layer L while entangling the muscle layer portion inverted and closed to prevent formation of a dead space.

Conventionally, the endoscope with a cap-type device attached to its distal end as described in Patent Document 1 could only resect the full thickness of a tubular wall that was large enough to fit into the cap-type device. According to the endoscopic treatment method according to the present embodiment, it is possible to resect the full thickness of a tubular wall of a larger size than the conventional one.

The practitioner finishes the EFTR technique by collecting the full thickness of the gastrointestinal wall that has been resected.

Although the first embodiment of the present disclosure has been described in detail above with reference to the drawings, the specific configuration is not limited to this embodiment, and design changes within the scope not departing from the gist of the present disclosure are also included. In addition, constituent elements shown in the above-described embodiment and in modification examples below can be combined and configured as appropriate.

### Second Embodiment)

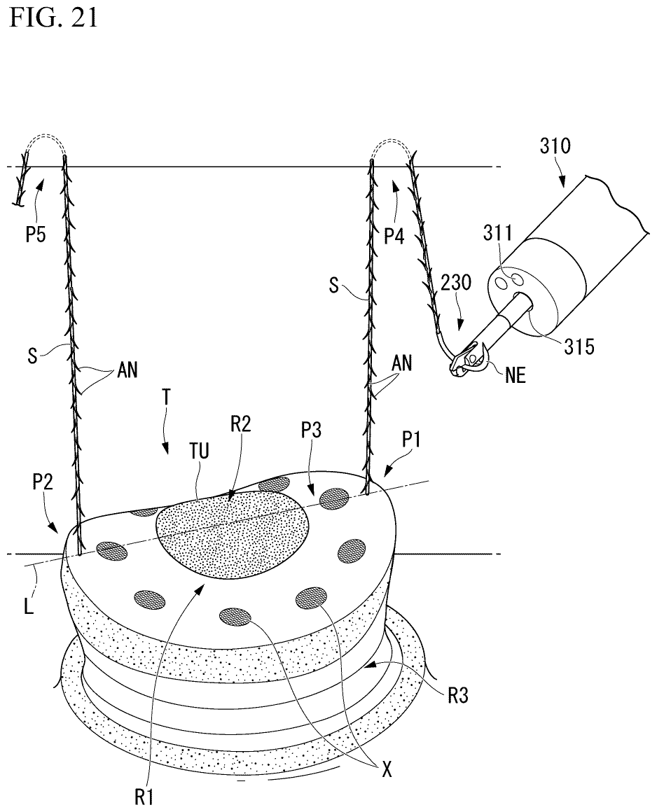

An endoscopic treatment method according to a second embodiment of the present disclosure will be described with reference to FIG. 21. In the following description, the configuration shared by that already described will be denoted by the same reference numerals, and the description thereof will not be repeated.

The endoscopic treatment method according to the present embodiment differs in the fixing step and the lifting step. Hereinafter, the fixing step and the lifting step of the endoscopic treatment method according to the present embodiment will be described.

### Fixing Step and Lifting Step

FIG. 21 is a view showing a fixing step and a lifting step.

A practitioner sews and fixes another needle-attached suture 400 to a second location P2 which is positioned further inside a part of a full circumferential incision site and further outside a lesion portion TU. The practitioner pierces the needle-attached suture 400 to a muscle layer P to penetrate the muscle layer P. The second location P2 is positioned distal to the lesion portion TU when viewed from an endoscope 300. The second location P2 may be a full circumferential incision site or a site where a submucosal layer N is exposed.

Next, the practitioner sews and fixes the needle-attached suture 400 to a first location P1 in the same manner as in the first embodiment. The practitioner pulls the first location P1 using the needle-attached suture 400 so that the first region R1 and the second region R2 are folded and approach each other, and lifts the full thickness of the gastrointestinal wall including the lesion portion TU toward the gastrointestinal lumen side. The practitioner fixes the needle-attached suture 400 to a fourth location P4 in the tissue of the gastrointestinal wall facing a target tissue TU.

The practitioner pulls the second location P2 using the needle-attached suture 400 so that the first region R1 and the second region R2 are folded and approach each other, and lifts the full thickness of the gastrointestinal wall including the lesion portion TU toward the gastrointestinal lumen side. Since the needle-attached suture 400 penetrates the muscle layer P, a mucosal layer L is less likely to be deviated with respect to the muscle layer P.

The practitioner fixes the needle-attached suture 400 to a fifth location P5 in the tissue of the gastrointestinal wall facing the target tissue TU. Since a suture S is provided with a barb (anchor) AN, it does not escape from the fifth location P5. Since a fixation tag (stopper) TG is provided at a proximal end of the suture S, the suture S does not escape from the second location P2 and is fixed to the second location P2. As a result, the second location P2 is connected to the fifth location P5 through the suture (connection member) S of the needle-attached suture 400. The fifth location P5 is desirably placed at a position facing the second location P2.

According to the endoscopic treatment method according to the present embodiment, by lifting the second location P2 in addition to the first location P1, a planned suture area R3 can be reliably elevated and exposed. Since the endoscopic treatment method according to the present embodiment elevates the target tissue T at two places, a large target tissue T can also be treated. It is desirable for the fourth location P4 and the fifth location P5 to be arranged at positions away from the center of the lesion portion TU. In addition, it is desirable for the distance between the fourth location P4 and the fifth location P5 to be longer than the distance between the first location P1 and the second location P2. This is because tension is likely to be applied to the center of the lesion portion TU and the entire lesion portion TU is likely to be retracted into the lumen. The practitioner may also attach the needle-attached suture (traction device) 400 to a third location P3 between the first location P1 and the second location P2 in addition to the first location P1 and the second location P2 to assist in lifting the target tissue T. The third location P3 may be a distal area of the planned suture area.

Although the second embodiment of the present disclosure has been described in detail above with reference to the drawings, the specific configuration is not limited to this embodiment, and design changes within the scope not departing from the gist of the present disclosure are also included. In addition, constituent elements shown in the above-described embodiments and in a modification example below can be combined and configured as appropriate.

### Third Embodiment

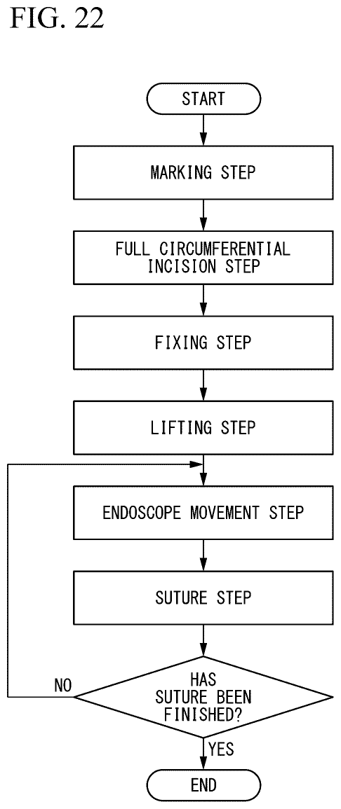

An endoscopic treatment method according to a third embodiment of the present disclosure will be described with reference to FIG. 22. In the following description, the configuration shared by that already described will be denoted by the same reference numerals, and the description thereof will not be repeated.

FIG. 22 is a flowchart of the endoscopic treatment method according to the present embodiment. The endoscopic treatment method according to the present embodiment differs in the sequence of treatment steps, and a suture step of performing suture and resection of the target tissue T is performed multiple times.

When suture and resection cannot be performed in one suture step, for example, when the target tissue T is large, a practitioner carries out the suture step multiple times. When the suture step is carried out multiple times, it is desirable for the practitioner to adjust tension of the suture (connection member) S connecting the first location P1 to the fourth location P4 by carrying out the lifting step in the middle of the resection as shown in FIG. 21. The shape of the target tissue T that has collapsed due to resection can be shaped so as to be folded along the fold line L. The practitioner may add the third location P3 as a lifting position in the middle of resection.

According to the endoscopic treatment method according to the present embodiment, a large target tissue T can also be treated by carrying out the suture step multiple times. The suture step can be suitably carried out by shaping the shape of the target tissue T in the middle of resection.

Although the third embodiment of the present disclosure has been described in detail above with reference to the drawings, the specific configuration is not limited to this embodiment, and design changes within the scope not departing from the gist of the present disclosure are also included. In addition, constituent elements shown in the above-described embodiments and in a modification example below can be combined and configured as appropriate.

What is claimed is:

1. An endoscopic treatment method for resecting a full thickness of a gastrointestinal wall under an endoscope, the method comprising:
fixing a traction device to a first location positioned further outside a target tissue;
pulling the first location using the traction device so that a first region and a second region are folded and approach each other along a fold line that passes through the first location and divides the target tissue into the first region and the second region, to lift the full thickness of the gastrointestinal wall including the target tissue toward a gastrointestinal lumen side;
suturing the lifted gastrointestinal wall positioned further outside the target tissue; and
resecting the target tissue placed in the lumen side of the sutured area.

2. The endoscopic treatment method according to claim 1, further comprising:
making a full circumferential incision in tissue around the target tissue up to a submucosal layer,
wherein the first location is further inside the incised site, and
wherein the sutured area of the gastrointestinal wall is the submucosal layer exposed by incision.

3. The endoscopic treatment method according to claim 1, further comprising:
moving the endoscope so that an endoscope optical axis is substantially parallel to the fold line after bringing the first region and the second region closer together and before suturing the submucosal layer.

4. The endoscopic treatment method according to claim 1, further comprising:
performing pressing so that the first region and the second region are in close contact with each other after lifting the target tissue using the traction device and before suturing the submucosal layer.

5. The endoscopic treatment method according to claim 4, further comprising:
performing pressing so that the first region and the second region are in close contact with each other using a pinching device capable of pinching tissue.

6. The endoscopic treatment method according to claim 5,
wherein the pinching device is a linear stapler or a clip.

7. The endoscopic treatment method according to claim 5,
wherein, when the target tissue is lifted by the traction device, the pinching device is movable regardless of the position of the traction device.

8. The endoscopic treatment method according to claim 1,
wherein suture and resection of the target tissue are performed multiple times.

9. The endoscopic treatment method according to claim 1, further comprising:
fixing the traction device through a muscle layer when the traction device is fixed to the first location.

10. The endoscopic treatment method according to claim 1,
wherein the first location is positioned proximal to the target tissue when viewed from the endoscope.

11. The endoscopic treatment method according to claim 10, further comprising:
lifting a second location positioned on a distal side when viewed from the endoscope in addition to the first location.

12. The endoscopic treatment method according to claim 11, further comprising:
attaching the traction device to a third location between the first location and the second location in addition to the first location and the second location to assist in lifting the target tissue.

13. The endoscopic treatment method according to claim 1, further comprising:
lifting the target tissue to a position where a muscle layer of the target tissue is sufficiently retracted to the gastrointestinal lumen side when performing lifting.

14. The endoscopic treatment method according to claim 1,
wherein the traction device is a needle-attached suture.

15. The endoscopic treatment method according to claim 1,
wherein the traction device includes at least one of a penetration member that penetrates tissue and a gripping member that grips the tissue, and a connection member connected to the one of them.

16. The endoscopic treatment method according to claim 15, further comprising:
fixing the traction device to a fourth location in tissue of the gastrointestinal wall facing the target tissue to connect the first location to the fourth location via the connection member.

17. The endoscopic treatment method according to claim 16, further comprising:
pulling the connection member to apply tension to the connection member that connects the first location to the fourth location.

18. The endoscopic treatment method according to claim 16,
wherein the fourth location is placed at a position facing the first location.

19. The endoscopic treatment method according to claim 16, further comprising:
applying tension to the connection member that connects the first location to the fourth location by delivering air endoscopically and dilating the lumen.

20. The endoscopic treatment method according to claim 16, further comprising:
adjusting a tension of the connection member that connects the first location to the fourth location in the middle of resection when resecting the target tissue.
